(19) United States
(12) Patent Application Publication
(10) Pub. No.: US 2022/0125953 A1
(43) Pub. Date: Apr. 28, 2022

(54) Synthetic Scaffolds for Metastasis Detection

(71) Applicant: Northwestern University, Evanston, IL (US)

(72) Inventors: Lonnie D. Shea, Chicago, IL (US); Samira M. Azarin, Chicago, IL (US); Robert M. Gower, Chicago, IL (US); Jacqueline Jeruss, Chicago, IL (US)

(21) Appl. No.: 17/516,289

(22) Filed: Nov. 1, 2021

Related U.S. Application Data
(63) Continuation of application No. 17/238,704, filed on Apr. 23, 2021, now abandoned.
Application No. 17/238,704 is a continuation of application No. 17/020,466, filed on Sep. 14, 2020, now abandoned.
Application No. 17/020,466 is a continuation of application No. 16/241,440, filed on Jan. 7, 2019, now abandoned.
Application No. 16/241,440 is a continuation of application No. 15/488,169, filed on Apr. 14, 2017, now abandoned.
Application No. 15/488,169 is a continuation of application No. 15/255,634, filed on Sep. 2, 2016, now abandoned.
Application No. 15/255,634 is a continuation of application No. 13/838,800, filed on Mar. 15, 2013, now abandoned.
(60) Provisional application No. 61/700,703, filed on Sep. 13, 2012.

(51) Int. Cl. A61K 49/00; A61L 27/18; A61L 27/54; A61L 27/56; C08L 67/04

(52) U.S. Cl. CPC A61K 49/0004; A61K 49/0008; A61K 49/0017; C08L 67/04; A61L 27/18; A61L 27/54; A61L 27/56; A61K 49/0063

(57) ABSTRACT

Provided herein are synthetic scaffolds engineered to function as a pre-metastatic niche to detect metastasis. In particular, synthetic scaffolds described herein provide detection of the earliest events in metastasis, thereby enabling treatment of metastasis before the disease burden increases.

### CROSS-REFERENCES TO RELATED APPLICATIONS

This application claims benefit of U.S. Provisional Application Ser. No. 61/700,703, filed Sep. 13, 2012, which is incorporated by reference herein in its entirety.

### FIELD

Provided herein are synthetic scaffolds engineered to function as a pre-metastatic niche to detect metastasis. In particular, synthetic scaffolds described herein provide detection of the earliest events in metastasis, thereby enabling treatment of metastasis before the disease burden increases.

### BACKGROUND

The discovery of metastatic spread of a primary tumor is often associated with poor prognosis, owing to the fact that the metastasis typically goes undetected until it has spread to the degree that it is affecting the function of one or more organs. Identification of metastasis prior to significant organ invasion would enable novel interventional strategies to halt disease progression while the disease burden is still low. Many technologies have been focused on screening for the presence of circulating tumor cells (CTCs) as a measure of metastasis, but these cells can remain in circulation for a long time before homing to and colonizing a metastatic site, with some tumor cells being shed very early in tumor progression.

### SUMMARY

This technology provides detection of the earliest events in metastasis, allowing treatment of metastasis before the disease burden is too high. Prior to the arrival of tumor cells at a metastatic site, other cell types arrive and set up an environment that promotes attachment and growth of the cancer cells. This environment is known as the “pre-metastatic niche.” In some embodiments, the present invention provides biomaterials and scaffolds and implants derived therefrom to engineer/create a “pre-metastatic niche” that is used to recruit and detect metastatic cells (e.g., in vitro, in vivo, in situ, etc.). In certain embodiments, the present invention provides a biomaterial scaffold and pre-metastatic niches engineered therefrom. Such scaffolds and pre-metastatic niches are used (e.g., in vivo) to recruit, detect, identify, and/or characterize metastatic cells. In certain embodiments, by enabling detection of cells that have acquired the ability to colonize a metastatic site, this technology provides a method of detecting the earliest events in metastasis. In some embodiments, this technology provides new methods of detecting and treating metastasis early in the progression of the disease.

In some embodiments, the present invention provides a biomaterial implant comprising a polymer scaffold and one or more chemical and/or biological agents, wherein the biomaterial implant mimics a pre-metastatic niche, recruits circulating metastatic cells, and provides an environment for metastasis. In some embodiments, the polymer scaffold is biodegradable. In some embodiments, the polymer scaffold is bioresorbable. In some embodiments, the polymer scaffold comprises poly(lactide-co-glycolide). In some embodiments, the one or more chemical and/or biological agents are selected from the list consisting of CD133, VEGFR-1, VEGFR-2, CD11b, GR1, F4/80, CD11b, CD11b+CD115+Ly6c+ and those listed in Table 1.

In some embodiments, the present invention provides methods of detecting metastasis in a subject comprising: (a) implanting a biomaterial implant into a subject; and (b) monitoring the biomaterial implant and/or the surrounding environment for changes indicative of metastasis. In some embodiments, the implant is implanted into a likely location of metastasis. In some embodiments, the location is selected from the list consisting of: lung, liver, brain, bone, and lymph nodes. In some embodiments, monitoring comprises inverse-scattering optical coherence tomography.

In some embodiments, the present invention provides methods of screening therapies for reducing, preventing, or treating tumor metastasis comprising: (a) exposing a test biomaterial implant of claim 1 to a putative therapy; (b) comparing test biomaterial implant to a control biomaterial implant; and (c) determining whether said therapy reduces, prevents, or treats tumor metastasis based on differences between said test and control biomaterial implants. In some embodiments, the test and control biomaterial implants are implanted in an animal. In some embodiments, the test and control biomaterial implants are implanted in the same animal. In some embodiments, the test and control biomaterial implants are implanted in the different animals. In some embodiments, the putative therapy comprises a pharmaceutical agent. In some embodiments, the differences between the test and control biomaterial implants comprise differences in the degree or amount of metastasis, cell recruitment, and/or colonization.

### BRIEF DESCRIPTION OF DRAWINGS

FIG. 1 shows a histogram of the number of migrated cells. MDA-MB-231 cells were serum starved for 24 h, then placed into the transwell migration assay suspended in regular or embryonic stem cell conditioned media. Cells were fixed and stained, and the number of migrated cells counted. Cell counts for cells not exposed to chemoattractant were used as control.

FIG. 2 shows microfabricated devices for analysis of directed cell movement. (a) Hoechst staining indicates cells throughout the top chamber and throughout the channel. (b), (c) Neurofilament staining that indicates axons elongating from the top chamber and down the channel. (d) Schematic of device for monitoring directed migration of metastic cells toward or away from the “niche.”

FIG. 3 shows a graph of average cell area. MDA-MB-231 cells were cultured in regular, embryonic stem cell conditioned, or mouse embryonic fibroblast conditioned media over a period of 10 days and average cell area was measured.

FIG. 4 shows bioluminescence Imaging (BLI) of live cell array. A) FLuc imaging B) Statistically significant normalized activity ratio. C) Correlation of TF activity between experiments performed on separate days.

FIG. 5 shows graphs of dynamic TF activity for MDA-MB-231 cells treated with stem cell conditioned media (red) or normal media (green). MDA-MB-231 breast cancer cells were infected with lentiviral firefly luciferase reporter constructs specific for shown transcription factors (TFs) and cultured for 8 days in BME. The activity of TFs was measured using bioluminescence imaging and normalized to basal TA promoter activity (TA-Fluc).

FIG. 6 shows images of PLG scaffold (dia=5 mm) and at 4× magnification, (B). Panel B indicates the random distribution of islets within the pores of the scaffold. (C) Delivery of Tregs with islets in an autoimmune model sustains normal blood glucose levels, indicating the localized prevention of islet destruction.

FIG. 7 shows images of detection of labeled breast cancer cells in vivo using IVIS imaging. (A) NSG mice inoculated with MDAMB-231 cells inoculated into the 4th MFP. (B) Image of metastases to the lung.

FIG. 8 shows (A) Bioluminescence image of a scaffold delivering a luciferase encoding plasmid. Transgene expression is localized to the implant site. (B) Quantification of luciferase levels through 150 days indicates relative stable transgene expression.

FIG. 9 shows graphs depicting IL10 expression and immune modulation. (A) Delivery of IL10 plasmid from the scaffold increases IL10 levels in the scaffold and the draining lymph node (DLN, right lumbar), but not in brachial lymph nodes or the blood. (B) The increased IL10 levels alters the trafficking of immune cells, as evidenced by the increased number of macrophages in the draining lymph node (DLN), but not in the non-draining (ND) left lumbar lymph node.

FIG. 10 shows images demonstrating blood vessel formation on PLG scaffolds with localized VEGF expression. Samples were retrieved 3 weeks post-implantation for (A) pLuc and (B) pVEGF. Scale bar, 2.5 mm. Immunohistochemical staining with CD31 (PECAM-1) monoclonal antibody to identify blood vessels (C). (D) Blood vessel density within tissue sections containing the polymer scaffold.

FIG. 11 shows graphs depicting: (a) Increase blood supply and the fractal dimension (D) of ECM ultrastructure measured by means of LEBS are markers of colon field carcinogenesis. Increased D was also confirmed in matrigel models conditioned malignant cells vs. control cells. (b)-(d): LEBS recorded from surrogate tissue sites senses mucosal alterations associated with field carcinogenesis and the presence of (pre)cancerous lesions elsewhere in an affected organ. (b) Ex vivo and in vivo colon cancer study (surrogate site—rectum). (c): Ex vivo and in vivo pancreatic cancer study (surrogate site—periampullary duodenal mucosa). (d): In vivo lung cancer study (surrogate site—buccal mucosa in the oral cavity).

FIG. 12 shows: (a) principles of ISOCT. ISOCT measures the auto-correlation function of tissue ultrastructure (length scales from 50 to 800 nm); (b) ISOCT 3D imaging of the fractal dimension D of macromolecular density in rectal mucosa; (c) Depth-profiles of D and correlation length scale lc; (d) Validation of the accuracy of ISOCT measurement of D in tissue models (suspension of nanospheres with fractal size distribution); (e) ISOCT confirmed an increase of D in colon field carcinogenesis. The graph shows an average difference between D in colon field carcinogenesis (rectal mucosa) vs. control rectal mucosa from no-neoplasia patients; (f) Schematic of a miniature ISOCT fiber-optic probe that will be developed as a potential solution in an unlikely case when a scaffold is implanted too deep to be non-invasively imaged by an open-air ISOCT system.

FIG. 13 shows whole animal bioluminescence imaging (BLI) of an NSG mouse containing a scaffold implant at day 23 post-tumor cell inoculation shows tumor cells are able to metastasize (A). White circle indicates location of scaffold implant. BLI of left peritoneal fat pads harvested at day 30 demonstrate that tumor cells are present in the peritoneal fat pad containing a scaffold (B) but absent in mice that did not receive a scaffold (C).

FIG. 14 shows flow cytometry analysis of scaffolds at day 14 post-tumor cell inoculation and day 7 post-implantation into the peritoneal fat pad. Leukocyte population dynamics within the scaffold are affected by the inclusion of a CCL22 viral vector (A). Accordingly, more tdTomato-positive tumor cells are recruited to the CCL22 scaffold (C) than the blank scaffold (B).

### DEFINITIONS

As used herein, the term “subject” refers to any human or animal (e.g., non-human primate, rodent, feline, canine, bovine, porcine, equine, etc.).

As used herein, the term “subject suspected of having cancer” refers to a subject that presents one or more symptoms indicative of a cancer or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis but for whom the stage of cancer is not known. The term further includes people who once had cancer (e.g., an individual in remission).

As used herein, the term “subject at risk for cancer” refers to a subject with one or more risk factors for developing a specific cancer. Risk factors may include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, metastasis of the cancer, and the subject's prognosis.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention.

As used herein, the term “initial diagnosis” refers to results of initial cancer diagnosis (e.g., the presence or absence of cancerous cells). An initial diagnosis does not include information about the stage of the cancer or the presence of metastasis.

As used herein the term “biodegradable” refers to a material (e.g., polymer) that breaks down into smaller or component parts (e.g., oligomeric and/or monomeric units) over a period of time (e.g., typically hours to months to years) when placed (e.g., implanted or injected) into a biological environment (e.g., into the body of a mammal).

As used herein, the term “bioresorbable” refers to a material (e.g., polymer), the degradative products of which are metabolized within or excreted from a biological environment (e.g., into the body of a mammal) within which they are placed, via natural pathways.

### DETAILED DESCRIPTION

Provided herein are synthetic scaffolds engineered to function as a pre-metastatic niche to detect and assess (e.g., evaluate efficacy of agents that reduce or prevent metastasis) metastasis. In particular, synthetic scaffolds described herein provide detection of the earliest events in metastasis, thereby allowing treatment of metastasis before the disease burden increases. Embodiments of the present invention provide detection of cells that have acquired the ability to colonize a metastatic site, as opposed to merely the presence of circulating tumor cells.

In some embodiments, the present invention provides a scaffold to provide a support for the attachment, colonization, growth, etc. or metastatic tumor cells. The scaffold provides a synthetic pre-metastatic niche, thereby mimicking conditions that allow for metastasis. In some embodiments, the scaffold is biodegradable and/or bioresorbable. In some embodiments, the scaffold is porous and/or permeable.

In certain embodiments, the scaffold comprises a polymeric matrix. In some embodiments, the matrix is prepared by a gas foaming/particulate leaching procedure, and includes a wet granulation step prior to gas foaming that allows for a homogeneous mixture of porogen and polymer and for sculpting the scaffold into the desired shape.

In some embodiments, the polymeric matrix in the scaffold acts as a substrate permissible for metastasis, colonization, cell growth, etc. In some embodiments, the scaffold provides an environment for attachment, incorporation, adhesion, encapsulation, etc. of chemical or biological agents (e.g., DNA, protein, cells, etc.) that create a pre-metastatic niche on and/or within the scaffold. In some embodiments, chemical and/or biological agents are released (e.g., controlled or sustained release) to attract circulating tumor cells, metastatic cells, or pre-metastatic cells.

In some embodiments, the biodegradable polymer is made by a gas foaming/particulate leaching process, as well as a wet granulation step, these methods thus avoiding the use of organic solvents and/or elevated temperatures and making it more conducive to incorporation of bioactive factors or cells. In some embodiments, particular combinations of high and low molecular weight polymers that, when combined with the wet granulation step prior to gas foaming and particulate leaching, allow for the sustained release of chemical and/or biological agents from the scaffold, and provides a mechanically stable scaffold which does not compress or collapse after in vivo implantation, thus providing proper conditions for cell growth.

In some embodiments, the polymer matrix used to prepare the scaffold comprises a biocompatible and biodegradable polymer. In yet another particular embodiment, the polymer matrix is a homopolymer or copolymer of lactic acid and/or glycolic acid and/or poly(caprolactone). In yet another particular embodiment, the polymer matrix comprises a homopolymer of a lactic acid or glycolic acid or poly caprolactone, a copolymer of a lactic acid and glycolic acid, or a copolymer of a lactic acid and a poly caprolactone, or a copolymer of a glycolic acid and poly caprolactone, or a copolymer of glycolic acid, lactic acid and a poly caprolactone. In yet another particular embodiment, the polymer matrix further comprises an aliphatic polyester, a polyanhydride, a polyphosphazine, a polyvinyl alcohol, a polypeptide, an alginate, or any combination thereof.

Scaffolds of the present invention may comprise any of a large variety of structures including, but not limited to, particles, beads, polymers, surfaces, implants, matrices, etc. Scaffolds may be of any suitable shape, for example, spherical, generally spherical (e.g., all dimensions within 25% of spherical), ellipsoidal, rod-shaped, globular, polyhedral, etc. The scaffold may also be of an irregular or branched shape.

In some embodiments, a scaffold comprises nanoparticles or microparticles (e.g., compressed or otherwise fashioned into a scaffold). In various embodiments, the largest cross-sectional diameters of a particle within a scaffold is less than about 1,000 μm, 500 μm, 200 μm, 100 μm, 50 μm, 20 μm, 10 μm, 5 μm, 2 μm, 1 μm, 500 nm, 400 nm, 300 nm, 200 nm or 100 nm. In some embodiments, a population of particles has an average diameter of: 200-1000 nm, 300-900 nm, 400-800 nm, 500-700 nm, etc. In some embodiments, the overall weights of the particles are less than about 10,000 kDa, less than about 5,000 kDa, or less than about 1,000 kDa, 500 kDa, 400 kDa, 300 kDa, 200 kDa, 100 kDa, 50 kDa, 20 kDa, 10 kDa.

In some embodiments, the scaffold is composed of material which is biodegradable and/or biorespobable. In some embodiments, the scaffold comprises a polymer. Suitable polymers include, for example, a polymer from the linear polyester family, such as polylactic acid, polyglycolic acid or polycaprolactone and their associated copolymers, e.g. poly(lactide-co-glycolide) at all lactide to glycolide ratios, and both L-lactide or D,L lactide. Polymers such as polyorthoester, polyanhydride, polydioxanone and polyhyroxybutyrate may also be employed.

In some embodiments, a scaffold comprises PLG. In some embodiments, PLG polymer is composed of 50:50 D,L-lactide:glycolide, 65:35 D,L-lactide:glycolide, 75:25 D,L-lactide:glycolide, 85:15 D,L-lactide:glycolide, D,L-lactide alone, L-lactide alone, 25:75 D,L-lactide:ε-caprolactone, 80:20 D,L-lactide:ε-caprolactone, ε-caprolactone alone, or other suitable formulations (e.g., other ratios between 99:1 and 50:50, other polymer combinations, etc.). In certain embodiments, PLG polymers are terminated by a functional group of chemical moiety (e.g., ester-terminated, acid-terminated, etc.). In some embodiments, PLG is modified (e.g., with poly(ethylene glycol)).

In some embodiments, the charge of a matrix material (e.g., positive, negative, neutral) is selected to impart application-specific benefits (e.g., physiological compatibility, beneficial interactions with chemical and/or biological agents, etc.). In certain embodiments scaffolds are capable of being conjugated, either directly or indirectly, to a chemical or biological agent). In some instances, a carrier has multiple binding sites (e.g., 2, 3, 4, 5, 6, 7, 8, 9, 10 . . . 20 . . . 50 . . . 100, 200, 500, 1000, 2000, 5000, 10,000, or more).

In some embodiments, one or more chemical and/or biological agents are associated with a scaffold to establish a hospitable environment for metastasis. Agents may be associated with the scaffold by covalent or non-covalent interactions, adhesion, encapsulation, etc. In some embodiments, a scaffold comprises one or more biological or chemical agents adhered to, adsorbed on, encapsulated within, and/or contained throughout the scaffold. The present invention is not limited by the nature of the chemical or biological agents. Such agents include, but are not limited to, proteins, nucleic acid molecules, small molecule drugs, lipids, carbohydrates, cells, cell components, and the like. In some embodiments, two or more (e.g., 3, 4, 5, 6, 7, 8, 9, 10 . . . 20 . . . 30 . . . 40 . . . , 50, amounts therein, or more) different chemical or biological agents are included on or within the carrier. In some embodiments, biological agents associated with a scaffold include metastatic markers, such as: CD133 (which generally defines all progenitors), VEGFR-1 (hematopoietic progenitor cells (HPCs)), VEGFR-2 (endothelial progenitor cells (EPCs)), CD11b and GR1 (myeloid-derived suppressor cells,12, 13), F4/80 and CD11b (macrophages14), and CD11b+CD115+Ly6c+ (inflammatory monocytes,15). In some embodiments, biological agents associated with a scaffold include factors that are active in the metastatic process, such as those listed in Table 1.

In some embodiments, agents are configured for stable association with the scaffold. In some embodiments, agents are configured for specific release rates. In some embodiments, multiple different agents are configured for different release rates. For example, a first agent may release over a period of hours while a second agent releases over a longer period of time (e.g., days, weeks, months, etc.). In some embodiments, the scaffold or a portion thereof is configured for slow-release of biological or chemical agents. In some embodiments, the slow release provides release of biologically active amounts of the agent over a period of at least 30 days (e.g., 40 days, 50 days, 60 days, 70 days, 80 days, 90 days, 100 days, 180 days, etc.). In some embodiments, the scaffold or a portion thereof is configured to be sufficiently porous to permit metastasis of cells into the pores. The size of the pores may be selected for particular cell types of interest and/or for the amount of ingrowth desired.

In some embodiments, the present invention provides methods and devices for detection of metastasis on an implanted scaffold. In some embodiments, non-invasive methods or metastasis detection are provided. In some embodiments, adapt inverse-scattering optical coherence tomography (ISOCT) is provided for non-invasive scaffold imaging. In certain embodiments, ISOCT enables three-dimensional (3D) imaging of tissue microvasculature and ultrastructure with detail that enables detection of metastasis to, upon, or within scaffolds. In some embodiments, the present invention is not limited by the methods, techniques, devices, etc. used for detection of metastasis to scaffolds.

In some embodiments, compositions and methods of the present invention provide a sensor of metastasis is a subject (e.g., a subject suspected of having cancer, a subject with cancer, a subject in remission, a subject not necessarily at elevated risk of cancer or metastasis). In some embodiments, a compositions is implanted within a subject and metastasis thereto is monitored to detect metastasis within the subject. In some embodiments, a device is implanted and checked at regular (e.g., daily, semi-daily, weekly, etc.) or periodic intervals (e.g., weekly monthly, yearly, etc.) for evidence of metastasis. In some embodiments, a single device is monitored over time for changes in the metastatic state thereof. In some embodiments, devices are implanted and removed following procedures to detect metastasis.

In some embodiments, the present invention provides compositions, methods, and assays for identifying therapies for preventing metastasis. In some embodiments, scaffolds are placed under conditions suitable for metastasis, and compositions, compound libraries, therapies, conditions, devices, etc. are tested for the ability to prevent and/or reduce metastasis.

### EXPERIMENTAL

### Example 1

### Identification of Microenvironment Signals that Promote Homing and Colonization of Metastatic Cells

Experiments have been conducted during development of embodiments of the present invention to identify the biological cues (e.g., within the pre-metastatic niche) involved in recruitment or metastatic cells, such as the cellular components (progenitor cells, immune cells), chemokines, and extracellular matrix proteins.

### A. Homing

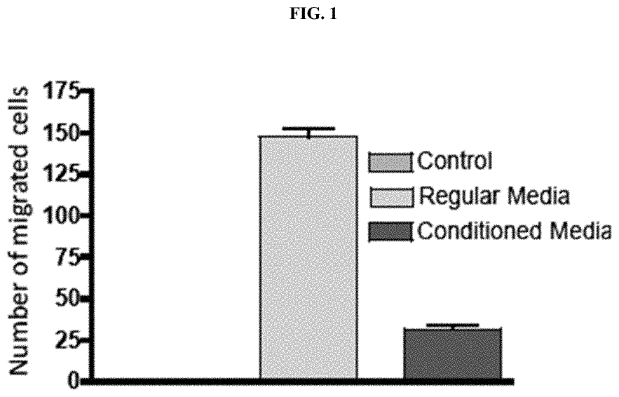

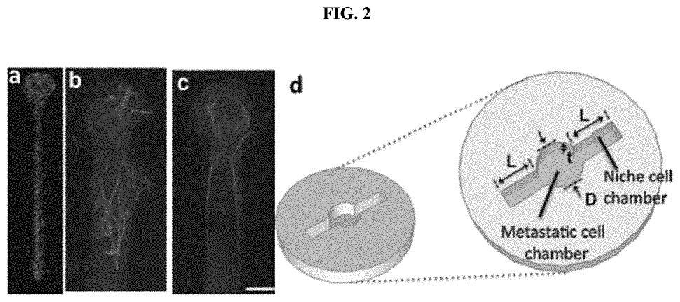

A function of the pre-metastatic niche is to induce metastatic cells homing. Factors secreted by cells within this niche can direct these metastatic cells. The traditional approach for investigating cell migration is a Boyden chamber. Experiments have been performed during development of embodiments of the present invention using a Boyden chamber to study the migration of highly metastatic MDA-MB-231 cells in response to factors produced by embryonic stem cells. Using a Boyden chamber, the influence of stem cells and their secretion of metastatic cells (SEE FIG. 1), with the exposed metastatic cells having a decreased migration rate. This assay can be adapted using a microfluidics system to allow for a determination of migration rate, and can also allow for the assessment of directed migration either toward or away from the source. This system can also determine a distance over which the cells respond to secreted factors, which can identify the gradient and concentrations necessary for promoting directed migration (SEE FIG. 2). Such microfluidics approaches have been used to investigate the directed extension of neurites, with extension occurring in response to secreted neurotrophic factors (J. A. Shepard, A. C. Stevans, S. Holland, C. E. Wang, A. Shikanov, and L. D. Shea, “Hydrogel design for supporting neurite outgrowth and promoting gene delivery to maximize neurite extension,” Biotechnol Bioeng (2011); herein incorporated by reference in its entirety). To investigate the homing of metastatic cells, the design illustrated in FIG. 2D can be employed, in which metastatic cells are placed in the central chamber and bone marrow derived cells (BMDCs) are placed in the niche compartment. The dimensions of the compartments are based on previous report that indicated directed motion up to a distance of 1.5 mm from the cell source. The length of the central compartment can be varied to assess the distance over which directed migration can be achieved. All cells can be encapsulated within Basement Membrane Extract (BME) (e.g., Matrigel) to investigate migration through a 3-dimensional matrix. Factors secreted by BMDCs within the niche compartment can create a gradient into the metastatic cell compartment. Homing is observed as the migration of the metastatic cells into the niche compartment, whereas repulsion could be observed as movement away from the niche.

The migration of the metastatic cells in the presence of the niche cells can be monitored by microscopy, as the metastatic cells are expressing fluorescent reporters. For the niche compartment, BMDCs can be obtained from the femurs of tumor bearing NSG mice, and the cells are sorted using markers such as, but not limited to, CD133 (which generally defines all progenitors), VEGFR-1 (hematopoietic progenitor cells (HPCs)), VEGFR-2 (endothelial progenitor cells (EPCs)), CD11b and GR1 (myeloid-derived suppressor cells,12, 13), F4/80 and CD11b (macrophages14), and CD11b+CD115+Ly6c+ (inflammatory monocytes,15). These cell types are listed as they have been associated with metastatic sites; however, additional immune cell populations as well as cells derived from other “niches” (e.g., stem cell niche bone marrow niche) are also evaluated. The metastatic cells (e.g., derived from human breast cancer) are seeded into chamber 2. The metastatic cell line MDA-MB-231-BR (231-BR) is a spontaneously metastasizing variant of the triple negative breast cancer line 231, which had previously undergone selection for its ability to metastasize to lung. After completion of the studies with the 231-BR cells, results are confirmed using primary human cells. Defined serum-free culture conditions will be used that are available for BMDCs and myeloid cells, which facilitates the identification of secreted factors by the “niche” cells. Time lapse images capture the maximal and mean distance that metastatic cells migrate toward or away from the “niche.”

These studies identify the cell types or combinations that maximally induce homing of metastatic cells, and these cells are subsequently employed to identify the factors associated with maximal homing. The function of the distinguished factors is investigated using a control cell (e.g. HEK293), which is transfected to expression the factor, with assessment of migration as described.

### B. Colonization

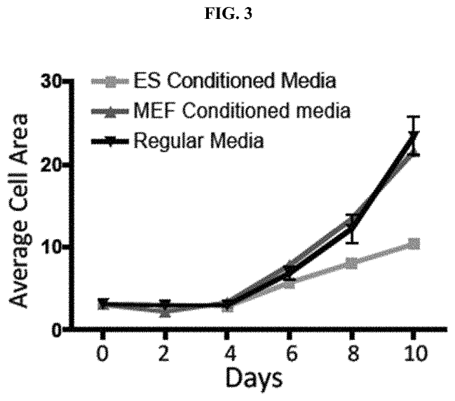

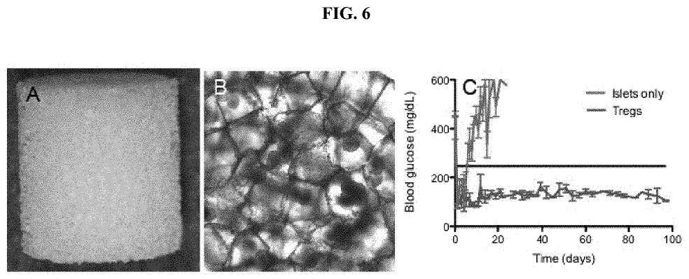

The environment of the pre-metastatic niche shows support the adhesion and growth of metastatic cells. Experiments are conducted to characterize the relative proliferation of metastatic cells as a function of the environment, namely the extracellular matrix composition and the presence of nich cells. The FDA approved biodegradable material pol (lactide-co-glycolide) (PLG) is used as a substrate for the homing metastatic cells, as this material can be modified with extracellular matrix (ECM proteins and can support the culture of multiple cell types). Additionally, this material is used for the in vivo studies. The ECM proteins to be used include, but are not limited to collagen (types I, IV), fibronectin, laminin, and BME, all of which have been implicated in the transition from dormancy to growth of cancer cells (Barkan, J. E. Green, and A. F. Chambers, “Extracellular matrix: a gatekeeper in the transition from dormancy to metastatic growth,” Eur J Cancer 46(7), 1181-1188 (2010); herein incorporated by reference in its entirety). Studies with MDA-MB-231 cells cultured in BME indicated that factors produced by ES cells inhibit cell proliferation (SEE FIG. 3). A similar analysis of proliferation is performed by co-culture of the metastatic cells with the BMDCs. Experiments employ a biomaterial disk on which the ECM proteins are adsorbed and the cell types seeded. Studies are performed with direct co-culture, as well as using conditioned media from the BMDC, which has the potential to distinguish between paracrine effects (e.g., conditioned media) and direct cell-cell contact or matrix deposition and remodeling. The disk conformation facilitates rapid analysis of cell proliferation using automated counting of the fluorescent cells. Follow-up studies are performed by seeding the cells onto microporous scaffolds (SEE FIG. 6A,B). Conditions that maximize proliferation of metastatic cells are investigated in to identify the key factors.

C. Cell Array and Proteomics to Identify the Factors that Drive Homing and Colonization

The identification of the factors driving homing and colonization is accomplished using two complementary approaches: i) a cell array for large scale analysis of transcription factor activity within the metastatic cells, with the active TFs connected to signaling pathways using bioinformatics tools. Computational techniques can identify the key signaling pathways associated with in vitro homing and colonization, and ii) a proteomics approach to identify the proteins secreted by the “niche” cells. A proteomics approach alone is contemplated to identify hundreds of proteins; however, this list of proteins can be cross-referenced with the key signaling pathways in order to identify those proteins that are most likely to induce the phenotype.

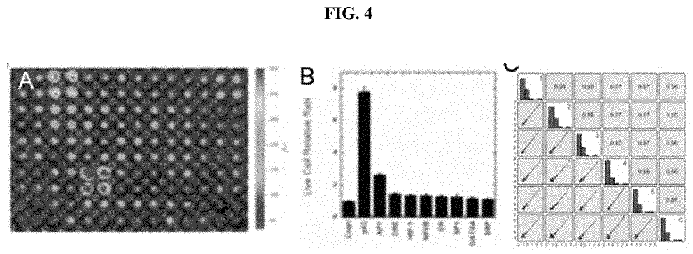

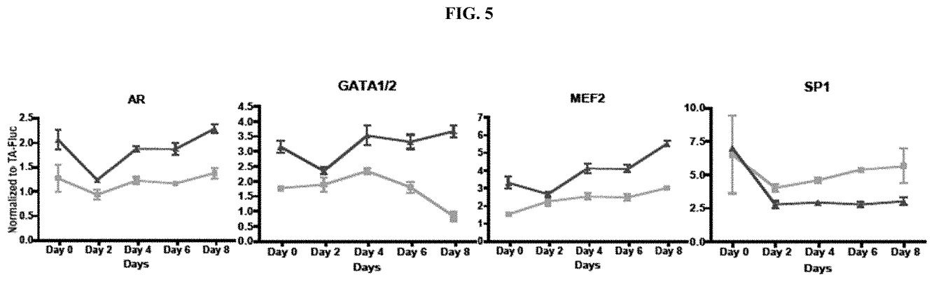

The metastatic cells, which is co-cultured with the “niche” cells, is analyzed for dynamic TF activity to identify signaling pathways critical to the metastatic process. Measuring the TF activity is accomplished using reporter constructs, in which a TF binding site modifies the basal TA promoter to induce firefly luciferase (FLuc) expression. Each well of the 384 well plate receives a distinct reporter construct, thus each well reports on the activity of a distinct TF (SEE FIG. 4 and Table 1). TF constructs are delivered as lentiviral vectors, and luciferase will be quantified by bioluminescence imaging (BLI). Constructs into which TF binding sites can be readily cloned, with binding sites identified from literature reports with established reporters, available Chip-seq data, or consensus sequences from TRANSFAC, the transcription factor database. The imaging approaches allow TF activity to be monitored in living cells, with light emission followed for multiple days (SEE FIG. 5). The consistency and reproducibility of the system has been established (M. S. Weiss, B. Penalver Bernabe, A. D. Bellis, L. J. Broadbelt, J. S. Jeruss, and L. D. Shea, “Dynamic, large-scale profiling of transcription factor activity from live cells in 3D culture,” PLoS One 5(11), e14026 (2010); herein incorporated by reference in its entirety). Images are captured at least three times per day for the duration of culture. This time course allows monitoring of changes in TF activity, and thus identifies initial effects of the drug and the ripple effect through the network.

TABLE 1

| | Associated | | Associated |
|---|---|---|---|
| | Transcription | | Transcription |
| Cell process | Factor | Cell process | Factor |
| Apoptosis | AP1, AP4, AR, | Differentiation | CREB, ER, |
| | BACH2, CREB, | | GATA1, GATA2, |
| | E2F, ELK1, ER, | | GATA3, GATA4, |
| | ETS1, ETS2, | | HES1, HIF1, |
| | FOXA2, FOXO3, | | IRF1, MAX, |
| | GLI1, GR, | | MYB, MYC, |
| | HDAC1, HIF1, | | NFAT, NFkB, |
| | HOXA1, HSF, | | NOTCH, RAR, |
| | IRF1, MAX, | | RXT, SMAD2, |
| | MEF2A, MEN1, | | SMAD3/4, |
| | MLL, MYB, | | SMAD6, SRF, |
| | NFkB, NOTCH1, | | STAT1, STAT3, |
| | p53, PIAS1, | | STAT4, VDR, |
| | PTTG, RELA, | | WT1, YY1 |
| | REST, RXR, | | |
| | SMAD2, | | |
| | SMAD3/4, | | |
| | SMAD6, STAT1, | | |
| | STAT2, STAT3, | | |
| | STAT6, VDR, | | |
| | WT1, YY1 | | |
| Cell Cycle | AP2, AR, E2F, | Transformation | AP1, BRCA1, |
| | ESX1, FOXO3, | | E2F, ER, ETS1, |
| | GR, HES1, KLF4, | | FOXO3, GLI1, |
| | MYB, MYC, | | GLI2, GR, |
| | NOTCH1, p53, | | HDAC1, MAX, |
| | PR, PTTG1, | | MYB, MYC, |
| | MSAD3/4, SP1, | | NFkB, NOTCH, |
| | WT1, YY1 | | p53, PAX3, TTG1, |
| EMT | LEF1, PAX3, | | RELA, STAT1, |
| | SMAD3/4, SP1, | | STAT2, WT1, |
| | STAT5, TCF3, | | YY1 |
| | TWIST, ZEB | | |
| Anchorage | AP1, FOXA2, | Metastasis | AP2, ER, ETS, |
| Independence | FOXO3, GR, | | GLI, KLF4, LEF, |
| | HES1, KLF4, | | MYC, OCT4, p53, |
| | MYC, NANOG, | | p63, p73, STAT1, |
| | NOTCH, PAX3, | | STAT3 |
| | PTTG1, RELA, | | |
| | WT1 | | |
| Inflammation | IRF1, MEF2A, | Migration | AP2, ER, ETS1, |
| | NFkB, PTTG1, | | HIF1, KLF4, |
| | RELA, SMAD3/4, | | NFAT, REST, |
| | STAT6, ZEB | | SRF, STAT3, |
| | | | TWIST |

To create the array, 231-BR cells are spinoculated with lentiviral reporter vectors placed into culture, the cells are deposited within the well plate with approximately 10,000 cells per well. The TF activity is calculated for each construct and statistical analysis is performed to identify the TFs with activity that is significantly above control, varies dynamically or between conditions. Data will be log transformed to assure the independence of the mean and the variance of the measurement. An Empirical Hierarchical Bayesian Method (EHBM), frequently used in high-throughput technologies such as microarrays, with Empirical Bayesian Hyperparameters that are calculated using Smyth's method is used to compare the individual TF activity relative to control, to assess differences between conditions, across arrays and experimental days. The final p-values will be corrected by the False Discovery Rate procedure, to reduce false positives and thus artifacts in the data. Results from the same condition performed in multiple experiments are combined using a modified meta-analysis procedure (D. V. Zaykin, L. A. Zhivotovsky, P. H. Westfall, and B. S. Weir, “Truncated product method for combining P-values,” in Genet Epidemiol, (2002), pp. 170-185; herein incorporated by reference in its entirety). Other methods to assess differential activity that account for the dependency of temporal data can also be incorporated, such as timecourse, EDGE, or BETR, widely used in microarray analysis of time series. Selected readings with significant activity are validated by quantification of luciferase using lysed cell assays, PCR, and shRNA knockdown. shRNA constructs are obtained from libraries at Open Biosystems. Additionally, the activity of key intermediates is analyzed using Luminex assays or phospho-Westerns. A partial least square regression (PLSR) analysis is applied to rank the TFs for their impact on the cell response. The key TFs are analyzed using existing bioinformatics databases to i) identify signaling pathways that are associated with the key TFs. The ligands for receptors that signaling through these pathways are cross-referenced with the proteomics analysis.

### Example 2

### Design of Biomaterial Implants that Promote Homing and Colonization of Metastatic Cancer Cells

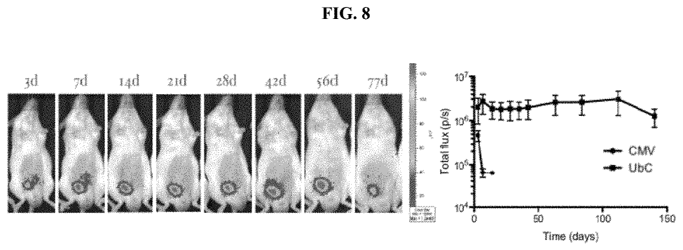

Experiments have been, and are, conducted during development of embodiments of the present invention to design/manufacture a synthetic environment (e.g., scaffold) that would compel metastatic cells to preferentially colonize the implant instead of other in vivo sites such as lung, liver, bone or brain. In some embodiments, a central feature of this approach is a porous polymer scaffold (SEE FIG. 6). The pores allow, for example, for cell infiltration and vascularization of the scaffold following implantation. BMDCs cells found in the pre-metastatic niche are naturally recruited to an implant site, and the ability to control the local environment within the scaffold can influence the distribution of cell types present. The local environment within the scaffold can be controlled by: i) extracellular matrix proteins immobilized to the material that influences cell infiltration, ii) cells seeded onto the scaffold and subsequently transplanted to influence the local environment through secreted factors, such as trophic factors or matrix proteins (SEE FIG. 6), and iii) gene therapy vectors associated with the scaffold that induce the localized expression of specific factors (SEE FIGS. 8,9). Using gene delivery as a means to modulate the local environment is attractive because expression can persist for long time periods, and the ability to modulate the target gene without needing to redesign the delivery system allows a variety of factors or combinations of factors to be investigated quickly.

### A. Transplantation of BMDCs Subsets to Promote Homing and Colonization by Metastatic Cells.

BMDCs are key to the process of recruiting of metastatic cancer cells. CD11b positive BMDCs have been demonstrated by multiple groups to be essential for seeding the pre-metastatic niche. Other subsets of BMDCs can support tumor progression and metastasis through regulating cellular processes such as angiogenesis, inflammation and immune suppression (B. Psaila, R. N. Kaplan, E. R. Port, and D. Lyden, “Priming the ‘soil’ for breast cancer metastasis: the pre-metastatic niche,” Breast Dis 26, 65-74 (2006); herein incorporated by reference in its entirety). A number of BMDCs, such as macrophages, myeloid derived suppressor cells, and endothelial progenitor cells, have been implicated in establishing a pre-metastatic niche that supports the homing and colonization of metastatic cancer cells. The scaffold can provide a powerful approach to investigate the contribution of specific cell subtypes, which are not currently well understood. FACS sorting of BMDCs is used to investigate the contribution of each subset to the recruitment of metastatic cancer cells.

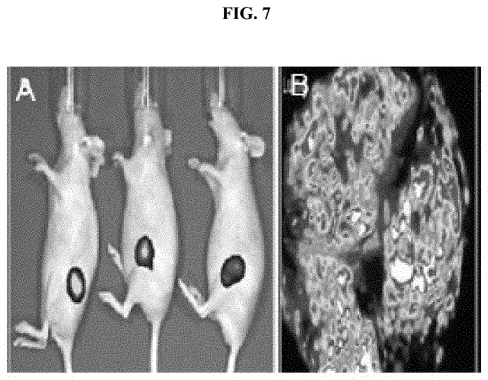

BMDCs are obtained from the femurs of tumor bearing NSG mice, and the cells are sorted for the desired markers listed in Aim 1. These cells are seeded onto the scaffolds (107 cells/mL) for subsequent implantation subcutaneously. Scaffolds (e.g., 5 mm diameter×2 mm height) are implanted subcutaneously, and 231-BR will be inoculated into the 4th mammary fat pad (MFP) 1 week post scaffold implantation. The subcutaneous site was selected for the following properties i) readily accessed, ii) ability to achieve long-term transgene gene expression by localized gene delivery from a scaffold, iii) the imaging system can be applied non-invasively. The 231-BR cells metastasize within 1-2 weeks. Follow-up studies are performed with primary metastatic breast cancer cells obtained with IRB approval. 231-BR cells do not normally metastasize to the subcutaneous space; thus providing the opportunity to create a site that uniquely recruits metastatic cancer cells. These cancer cells have been engineered to express both luciferase and mCherry allowing for non-invasive imaging of their in vivo distribution (SEE FIG. 7). Luminescence imaging of luciferase is used initially as it is more sensitive than fluorescence. Following cell transplantation, the scaffold is imaged 4× per week for 5 weeks in order to determine the extent to which cells metastasize to the scaffold. The BLI techniques provide a rapid, and quantitative assessment of the number of cells at a site, and also determine the relative numbers of cells within the scaffold relative to other sites (e.g., lung). For scaffold conditions that have maximal metastasis, tumors are weighed and the distribution of cell types within the graft are characterized, both transplanted and recruited, at 2 and 4 weeks. Samples are retrieved and analyzed by flow cytometry to determine the relative frequency of the BMDCs, and metastatic cells. 8-color flow cytometry is performed using CD11b, (macrophage and neutrophil progenitors) CD11c (dendritic cells), Ly6 (monocyte progenitors), VEGFR2 (endothelial progenitor cells), VEGFR1 (hematopoietic progenitor cells), GR1 (neutrophils), F4/80 (macrophage), and CD133. Additionally, the scaffolds are analyzed histologically to investigate the spatial distribution of the BMDCs, immune, and metastatic cells. Sections are immunostained for markers such as CD11b, CD11c, Ly6, VEGFR2, and VEGFR1. Tumor cells in sections are detected by fluorescence and tumor cell proliferation in the scaffold are evaluated using Ki67 immunostaining.

### B. Vectors Encoding for Cytokines to Modulate BMDC Recruitment

Scaffold implantation leads to a foreign body response that recruits BMDCs to the scaffold. Embodiments of the present invention modulate the recruitment of BMDCs through the localized expression of cytokines, which influence the phenotype of monocytes, macrophages, and dendritic cells (DCs) (Kaplan et al. Cancer Metastasis Rev 25(4), 521-529 (2007).; Kaplan et al. Nature 438(7069), 820-827 (2005).; Psaila et al., Breast Dis 26, 65-74 (2006).; herein incorporated by reference in their entireties). Expression of cytokines may convert endogeneously recruited macrophages or DCs into cells that subsequently recruit metastatic cells. Scaffolds are created and loaded with lentivirus encoding for either pro-inflammatory cytokines (TNF-alpha, IFN-gamma, IL-1β. IL-6, IL-8), or anti-inflammatory cytokines (TGFβ, IL-10, IL-4).

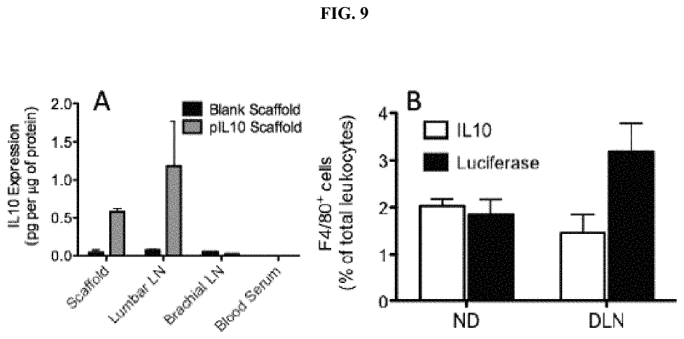

Scaffolds are implanted subcutaneously, with delivery of 231-BR inoculated into the MFP 1 week post scaffold implantation. Vectors encoding for pro-inflammatory cytokines (TNF-alpha, IFN-gamma, IL-1β. IL-6, IL-8), or anti-inflammatory cytokines (TGFβ, IL-10, IL-4), are locally delivered. The localized delivery of plasmid encoding for IL-10 leads to significant increases in IL-10 at the graft, and the draining lymph node (SEE FIG. 9A). Furthermore, an analysis of immune cell types at the draining lymph nodes indicates that the number of macrophages is significantly increased in the draining lymph node, indicating an altered trafficking of the immune cells through the graft (SEE FIG. 9B).

Experiments are conducted with scaffolds loaded with either an inflammatory or anti-inflammatory cytokine with imaging as the primary endpoint. BLI is employed with the scaffolds 4× per week for 5 weeks in order to determine the extent to which cells colonize the scaffold, as well as other sites (e.g., lung). For scaffold formulations with maximal metastasis, tumor weight, the relative frequency of cell types, and histology is analyzed at 2 weeks and 4 weeks. The analysis by flow cytometry and histology particularly focuses on BMDCs. Immunostaining is performed on these sections for CD11b, (macrophage and neutrophil progenitors) CD11c (dendritic cells), Ly6 (monocyte progenitors), VEGFR1 (endothelial progenitor cells), VEGFR-1 (hematopoietic progenitor cells). Tumor cells in sections are detected by fluorescence or luminescence.

C. Vectors Encoding for Factors that Promote the Homing and Colonization of Metastatic Cancer Cells

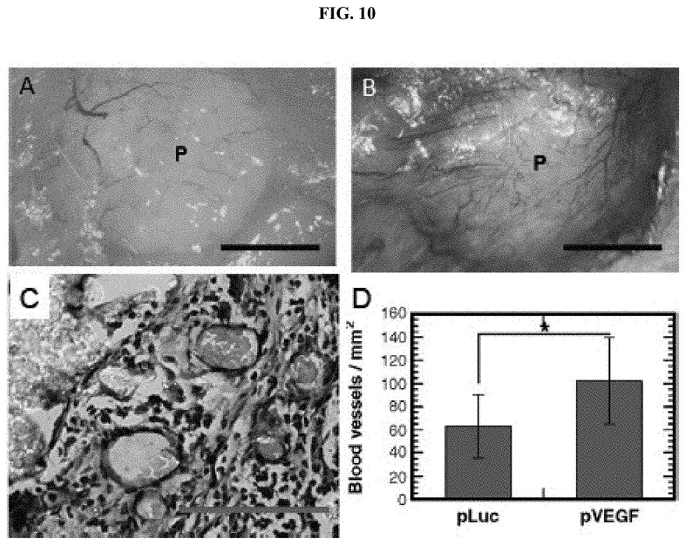

Experiments are conducted to analyze the localized expression of factors that have been implicated in direct recruitment of metastatic cells. CXCL12/SDF-1 is the ligand that binds to CXCR4, which is the most prominent chemokine receptor expressed on breast cancer cells and is not expressed on normal breast cells. CXCR4 is a major factor in breast cancer metastasis due to migration of the cancerous cells through CXCL12 signaling with surrounding tissues. Alternatively, VEGF, which is produced by multiple cell types, such as EPCs and macrophages, has been hypothesized to influence the homing of metastatic cancer cells. In addition to being secreted by these cell types, VEGF can be released from the matrix due to localized matrix degradation. Other factors (e.g., paracrine factors) identified during the experiments described herein, that influence metastatic cell homing and colonization, are included. Scaffolds loaded with vectors encoding SDF-1 or VEGF and cancer cells are delivered as described, as described above. The expression of VEGF following plasmid delivery from scaffolds has previously been reported to significantly enhance vessel growth into the scaffold (SEE FIG. 10). As described above, BLI, flow cytometry, and histology is employed to characterize the extent of metastatic cell homing.

### Example 3

### Non-Invasive Imaging

Some clinical applications require a non-invasive means of detecting the colonization of implanted scaffolds by metastatic cells in vivo. While whole-body imaging techniques are not able to provide the requisite resolution and sensitivity for detection of a few metastatic cells within a scaffold, the use of optical imaging is well suited for this application. A conventional approach (commonly employed in animal studies) would be to image the malignant cells as they colonize the scaffold by means of optical molecular imaging. Although powerful, this requires the malignant cells to be tagged with fluorescent labels. In clinical practice, however, it is difficult to know a priori what kind of molecular markers the colonizing malignant cells exhibit and thus molecular imaging is not be suitable. Therefore, embodiments described herein focus on endogenous markers of colonization. Since only a few malignant cells may be present in the scaffold, instead of attempting to detect the cells themselves, an alternate approach is utilized: detection of the effect of malignant cells on the scaffold, e.g., a change in the microenvironment caused by the colonizing cells. Light-scattering-based imaging provides for detection of subtle, microscopically undetectable alterations such as changes in the microvasculature and ultrastructure within the scaffold.

Experiments are conducted to adapt inverse-scattering optical coherence tomography (ISOCT) for non-invasive scaffold imaging in vivo. ISOCT enables three-dimensional (3D) imaging of tissue microvasculature and ultrastructure with detail well below the diffraction-limited limit of resolution (sensitivity to length scales as small as 40 nm). ISOCT is developed for non-invasive imaging of subcutaneously implanted scaffolds. ISOCT and scanning transmission electron microscopy (STEM) are performed ex vivo on scaffolds extracted after colonization and control scaffolds in order to identify ISOCT-detectable endogenous ultrastructural and microvascular markers of the scaffolds' response to cell migration. Experiments are conducted to determine the minimal number of malignant cells that induce a microenvironmental change detectable by ISOCT.

### Optical Detection of Alterations in Extracellular Matrix (ECM) in Carcinogenesis.

ECM is altered in tumors. Changes occur in the ECM of the mucosa in the earlier, pre-neoplastic stage of carcinogenesis including field carcinogenesis. Field carcinogenesis is a common theme in almost all carcinomas and is the notion that the genetic/environmental milieu that leads to a focal tumor exists not only at that particular location but affects the entire organ, and the molecular and nanostructural alterations that develop diffusely in an affected organ provide a fertile mutational environment with focal tumors occurring due to a stochastic mutational event.) Experiments have demonstrated that micro-vascular and ultrastructural alterations in colon, pancreatic and lung field carcinogenesis. An optical technique called low-coherence enhanced backscattering (LEBS) was used to probe mucosa close to the tissue surface (up to a few hundred microns) and does not have a 3D imaging capability. ISOCT has been developed to obtain the same information about tissue structure and microvasculature as LEBS does, while also affording a 3D imaging capability.

Two most pronounced—and consistent across different cancer types—ECM alterations have been observed in field carcinogenesis: an increase in the microvascular blood supply, which is induced in part by iNOS and due to both neovascularization and vasodilation, and an alteration in the auto-correlation function of macromolecular density B(r) for length scales from ˜ 1/15th (˜40 nm) of the wavelength of light λ to ˜λ. Specifically, LEBS measures the shape of B(r), D. If D<3, B(r) is a mass fractal with D being its mass fractal dimension, while 3<D<4 corresponds to a stretched exponential B(r), D=4 is exponential and D>4, Gaussian. Most types of ECM have fractal-like organization (i.e., B(r) is an inverse power-law for 40-800 nm length scales) and field carcinogenesis is associated with an increase in D. In the ECM, this most likely corresponds to changes in collagen matrix cross-linking.

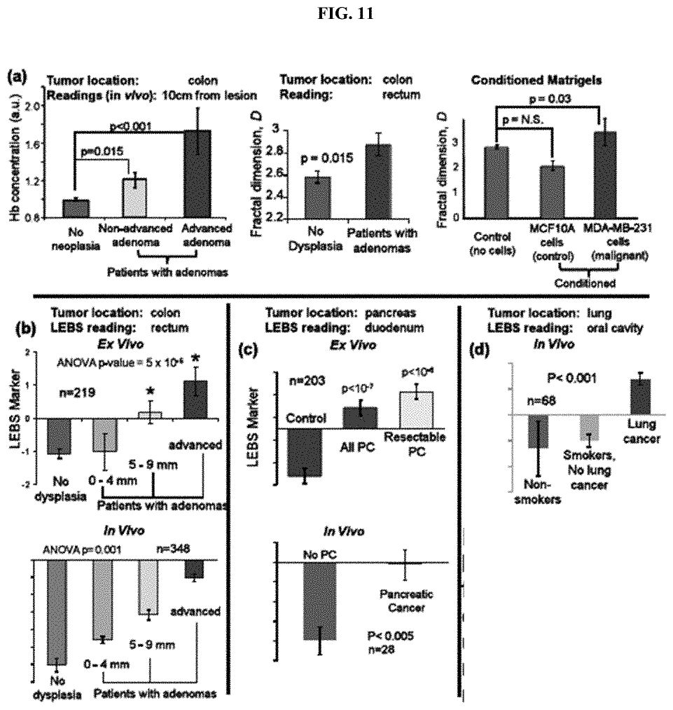

Colon field carcinogenesis: LEBS (penetration depth ˜100 μm) was recorded either ex vivo from histologically-normal rectal biopsies (n=419) or in vivo by means of a fiber-optic probe and compared with the outcome of colonoscopy: presence of adenomas anywhere in the colon. The observed increase in the mucosal microvascular blood supply and D paralleled the risk of developing colon cancer (SEE FIG. 11A/B). In a double-blinded in vivo validation study, an LEBS biomarker obtained as a linear combination of the two individual markers showed an excellent diagnostic performance differentiating patients who harbored advanced adenomas and those who were neoplasia-free irrespective of the location of the lesions (92% sensitivity, 74% specificity). Biomarkers were not confounded by demographic, risk factors or benign lesions. Pancreatic Cancer (PC): periampullary duodenal mucosa was assessed as a surrogate site for PC in patients with and without PC both ex vivo and in vivo. LEBS marker correlated with PC (SEE FIG. 11C) with 73% sensitivity and 89% specificity for resectable tumors. This was not confounded by demographic, risk factors, benign pancreatic pathologies (e.g. pancreatitis) and tumor location and stage. Lung cancer: buccal (oral cavity) mucosa was probed from smoking patients with and without lung cancer. LEBS performance in a prospective validation dataset was excellent (100% sensitivity, 70% specificity) and was not diminished for patients with early stage cancers, with no confounding by demographic factors or the amount of smoking (SEE FIG. 11D). Matrigels: An increase in D was confirmed in matrigel models conditioned by malignant cells (MDA-Mb-231) vs. those conditioned by non-malignant cells (MCF10A).

### A. Development of ISOCT

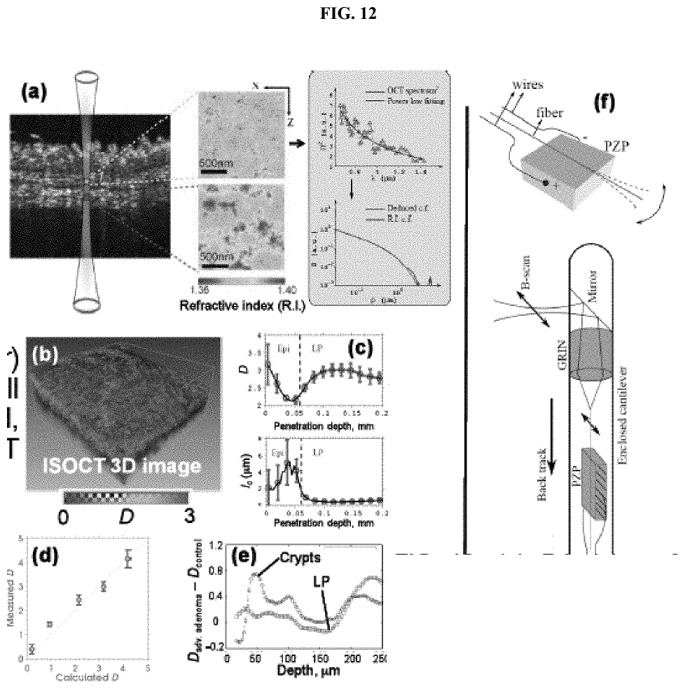

The thrust to understand nanoscale processes in tissue has been stymied by the lack of a practical means to image tissue structure at the nanoscale to the diffraction limit of resolution of existing imaging techniques. ISOCT was developed to address this need. ISOCT offers a label-free approach to quantify the statistical mass density correlation function of tissue with subdiffractional sensitivity. Compared to conventional OCT, ISOCT inverses the physical process of light scattering to quantify the physical properties of tissue at the nanoscale for each microscopic voxel of a 3D tissue image up to a few mm penetration depth (SEE FIG. 12A). As opposed to conventional approaches, ISOCT is sensitive to subdiffractional length scales because it does not attempt to visualize these small structures but instead quantifies their statistics via a spectral analysis.

ISOCT relies on the 3D spatial resolution conventional OCT and the spectral analysis of the sign recorded from each 3D voxel to measure the ultrastructural and microvascular properties of tissue. T analysis of tissue ultrastructure is based on the fact that contrast in ISOCT is due to light scattering by the spatial variations of the refractive index, and an ISOCT spectrum has a Fourier transform dependence on the au correlation function B(r) of the refractive index (an macromolecular density) for length scales r from 40 to 80 nm (λ/15<r<λ, similar to LEBS). Thus, the spectral analysis is used to estimate B(r). B(r) is the key physical characteristic of tissue ultrastructure. In practice, it convenient to model B(r) by the Whittle-Mattern family s that B(r) is fully described by three parameters: i functional form (D, see above), the average amplitude (Δn) and the length scale (lc) of the refractive index. For each voxel in tissue, D is derived from the spectral slope of an ISOCT spectrum and then Δn and lc are estimated from the scattering and backscattering coefficients of tissue, which are measured based on the intensity of ISOCT and its attenuation with depth. Thus, ISOCT generates 3D images of the ultrastructural parameters. FIGS. 12B and 12C illustrate the information obtained by ISOCT. We imaged rectal biopsies from subjects with and without colon adenomas. ISOCT showed that B(r) is a mass fractal with fractal dimension D increasing in field carcinogenesis (consistent with LEBS) (SEE FIG. 12E).

ISOCT is adapted for non-invasive imaging of scaffolds. (i) The existing version of the ISOCT instrument uses visible light and can image up to ˜500 μm deep into tissue. This range is extended to >900 nm (source: NKT SuperK supercontinuum laser, detector: SU-LDH2, Goodrich), which increases the maximal penetration depth up to 3 mm, sufficient for scaffold imaging. Although this slightly changes the range of length scales probed by ISOCT to 60-1000 nm, the majority of the diagnostic range is still be covered. (ii) An algorithm is developed for LEBS measurement of microvascular blood content. Hb has highly specific absorption bands in the visible and near-IR spectra. Fitting a modified Beer's law model that incorporates both absorption and scattering into LEBS spectra enabled measurement (±5-19% error depending on depth of penetration) of the total Hb content, oxygenation, and average blood vessel diameter. Experiments with tissue models, animal and human studies have proven the accuracy.

### B. Identify a Set of ISOCT-Detectable Biomarkers of Colonization

Based on preliminary data, microvascular and ultrastructural markers are focused on. Subcutaneously implanted scaffolds will be extracted from animals with and without tumors (power analysis (non-parametric Wilcoxon-Mann-Whitney test)) performed based on the coefficients of variability for ECM alterations in colon field carcinogenesis). Endpoints for each potential biomarker are its effect size between cases and controls (ratio of the absolute effect to the cumulative standard deviation), sensitivity and specificity. After the ISOCT analysis, cells are extracted, and the minimum concentration of colonizing cells sufficient to induce an ISOCT-detectable change in the scaffolds is determined.

Because B(r) is the fundamental descriptor of the ultrastructural alterations, a strategy is to measure B(r) of the scaffolds by high-resolution imaging (STEM) and identify the key alterations at length scales to which ISOCT is sensitive (>60 nm). At length scales above the 3D spatial resolution of ISOCT (lateral and axial resolution 1 and 10 μm), STEM is no longer needed and ISOCT itself will provide complete 3D imaging. High-angle annual dark-field STEM allows accurate mass determination with no contrast agents. By collecting scattered electrons for each pixel, a quantitative density map is obtained. The mass density images are converted to the spatially varying refractive index (which is a linear function of the mass density), and then input into the finite-difference time-domain (FDTD) simulations, which are numerically solve Maxwell equations and determine the differential scattering cross section and, via a newly developed software platform ANGORA that uses FDTD to accurately model essentially any time of microscopy, ISOCT signal. The STEM→FDTD-predicted and experimentally observed ISOCT alterations are compared. Knowing how B(r) changes in colonized scaffolds (from STEM imaging) allows identification of which other ISOCT parameters are affected, which is cross-validated by the experimental ISOC data.

A prediction rule based on the ISOCT-detectable biomarkers designed for high sensitivity is developed. Two sample t-tests will be used to compare cases vs. controls. Univariate logistic regression determines the significance of each marker. A multi-variable logistic regression assesses collinearity, confounding, and effect modifications. Possible interaction between covariates is evaluated. The final model includes statistically significant covariates. An ROC curve is made for each marker and the optimal cut-off points will be selected. Multi-variable logistic regression is used to formulate the rule. The reliability of the model is evaluated by the Hosmer-Lemeshow goodness-of-fit test. The statistical significance of the individual markers in the model is evaluated using the Wald test. The discriminative ability of the model is evaluated by the area under the ROC curve (AUC) and its 95% confidence level.

### C. In Vivo Validation of ISOCT Biomarkers

The ISOCT prediction rule is tested in vivo on an independent set of animals with subcutaneous implants with and without tumors at different time points (days 0-10 with a time point every other day). After the last time point, the scaffolds are extracted and colonization confirmed. Cut-points for the biomarkers are prospectively assessed. The endpoints of the in vivo validation are AUC, sensitivity and specificity of ISOCT and the earliest time point of detection. If sensitivity of ISOCT diminishes significantly even for practical depths of implantation, or if wind that subcutaneous implantation is suboptimal, a miniature ISOCT fiber-optic probe, under 1 mm in diameter, is developed to probe scaffolds that are otherwise not accessible (SEE FIG. 12F). While conventional OCT probes require a circular scan of the beam driven by an external rotor or an internal spiral track, those mechanical parts limit probe miniaturization. Instead, in order to keep the probe size under 1 mm, a scanning mechanism based on a piezo plate (PZP) actuator will be adopted. A single mode fiber branched from a fiber coupler (interferometer for OCT) is glued onto the plate. When a sinusoidal wave form is applied on the PZP, the fiber tip oscillates along the vibration direction and form a stable resonance scan (the B-scan in FIG. 12F). The PZP and the fiber tip are followed by a gradient-index (GRIN) lens that focuses the light and also collects the backscattered light. A reflective mirror is attached to the end of the GRIN lens to steer the light onto the tissue. All the miniature optics are enclosed in a cantilever, which is retracted at a set speed to realize the scan in the other lateral direction. Thus, a 3D image is generated after the retrieval of the cantilever and the ISOCT analysis is performed.

### Example 4

### Metastasis to Scaffold

Porous polymer scaffolds have been developed that are, for example, 5 mm in diameter and 2 mm in height and are composed of poly(lactide-co-glycolide) (PLG), an FDA-approved biodegradable material (for synthesis/manufacture of scaffolds, see, e.g., US Pat. Applications 2002/0045672, 2005/0090008, 2006/0002978, and 2009/0238879 (each of which is herein incorporated by reference in their entirety) and U.S. Pat. Nos. 7,846,466; 7,427,602; 7,029,697; 6,890,556; 6,797,738; and 6,281,256 (each of which is herein incorporated by reference in their entirety). The PLG microspheres are then mixed with 250-425 μm NaCl particles in a 30:1 ratio and pressed in a steel die at 1500 psi. The scaffolds are then gas-foamed and salt particles are removed by washing in water. Upon implantation of the exemplary scaffolds, cells from the host tissue and blood vessels infiltrate the scaffold. These scaffolds have also been used as vehicles for localized delivery of gene therapy vectors, which can produce long-term localized transgene expression. The delivery of vectors encoding diffusible factors have influenced cellular processes, with vectors encoding angiogenic factors enhancing vascularization and chemoattractants affecting immune cell infiltration. Finally, these scaffolds have been used as vehicles for cell transplantation, resulting in survival and localized function.

To demonstrate that such scaffolds are capable of attracting metastatic cells, an orthotopic xenotransplant model of human breast cancer metastasis in female NSG mice was developed. The metastatic human cell line used for studies conducted during development of embodiments of the present invention was MDA-MB-231-BR (231BR), a spontaneously metastasizing variant of the triple-negative MDA-MB-231 breast cancer line, which has previously undergone selection for its ability to metastasize to the brain. The 231BR cell line was then stably transfected to express luciferase and tdTomato to generate the 231BR-TOM-LUC2 cell line.

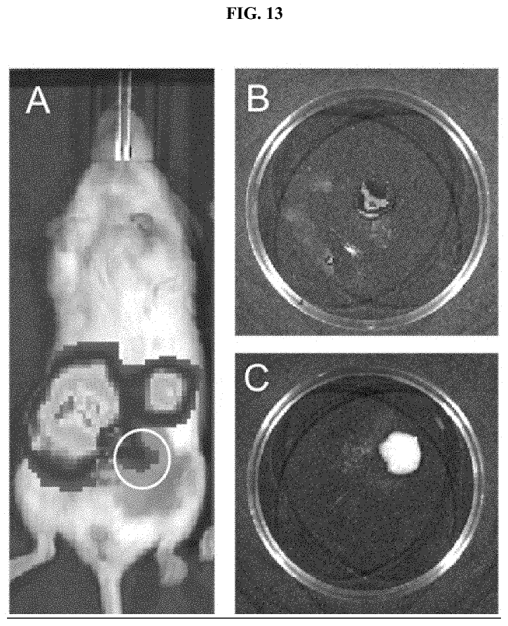

Tumor inoculation was performed by injecting 2E6 231BR-TOM-LUC2 cells in a volume of 50 μL into the right mammary fat pad of NSG mice, and 7 days post-inoculation, PLG scaffolds were implanted into the left peritoneal fat pad. Whole animal bioluminescence imaging (BLI) was performed twice weekly to track tumor growth and spatial distribution of 231BR-TOM-LUC2 cells. Metastasis to the area containing the scaffold can be visualized using BLI (FIG. 13A). Additionally, tumor cells metastasize to peritoneal fat pad if a scaffold is present (FIG. 13B) but not if the mouse did not receive a scaffold implant (FIG. 13C), indicating that the inflammatory response generated by implantation of the scaffold enables recruitment of metastatic cells to a site to which they typically do not metastasize. This data demonstrates that metastasis to the scaffold can be readily achieved and thus the scaffold technology can be used to create a controlled environment to recruit metastatic cells.

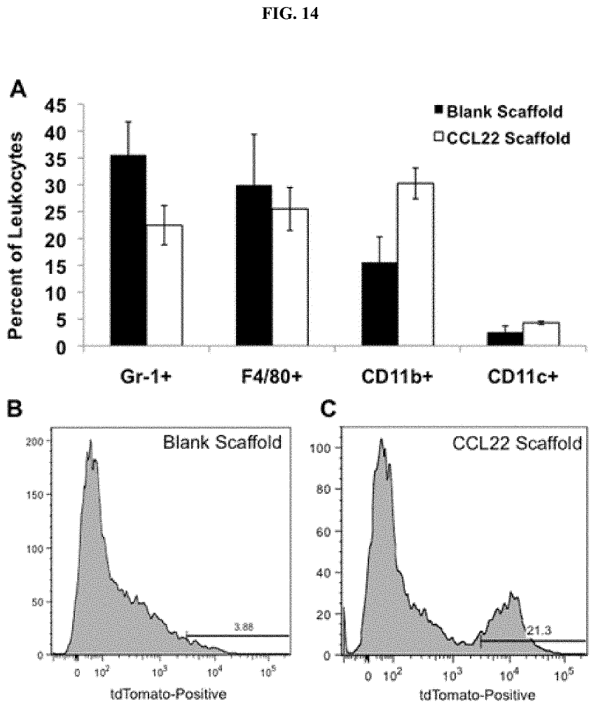

To demonstrate that the scaffold is further engineered to include signals that promote enhanced recruitment of metastatic cells, a viral vector encoding for expression of CCL22 was included in the scaffold. CCL22 expression modulates the immune cell populations present in the scaffold and thus affect establishment of the pre-metastatic niche and recruitment of metastatic cells. Flow cytometry was performed 7 days after the scaffolds were implanted in tumor-bearing mice to determine the effect of CCL22 on the percentage of different types of leukocytes present in the scaffold. FIG. 14A demonstrates that scaffolds with the CCL22 viral vector contained higher percentages of CD11b+ cells (monocytes) and lower percentages of Gr-1+ cells (neutrophils) than blank scaffolds that did not contain the CCL22 viral vector.

Additionally, the scaffolds containing CCL22 (FIG. 14C) were able to recruit a higher percentage of tdTomato-positive tumor cells than the blank scaffold (FIG. 14B). This data provides evidence that modulating the inflammatory cell populations present in the scaffold can influence recruitment of metastatic cells, indicating that the scaffold can be engineered to promote maximal recruitment of metastatic cells in order to develop detection techniques for early metastatic events and to study the signals involved in establishing the pre-metastatic niche.

Various modification, recombination, and variation of the described features and embodiments will be apparent to those skilled in the art without departing from the scope and spirit of the invention. Although specific embodiments have been described, it should be understood that the invention as claimed should not be unduly limited to such specific embodiments. Indeed, various modifications of the described modes and embodiments that are obvious to those skilled in the relevant fields are intended to be within the scope of the following claims. For example, U.S. Pat. Applications 2002/0045672, 2005/0090008, 2006/0002978, and 2009/0238879 (each of which is herein incorporated by reference in their entirety) and U.S. Pat. Nos. 7,846,466; 7,427,602; 7,029,697; 6,890,556; 6,797,738; and 6,281,256 (each of which is herein incorporated by reference in their entirety) provide details, modifications, and variations that find use in various embodiments described herein. All publications and patents mentioned in the present application and/or listed below are herein incorporated by reference in their entireties.

### REFERENCES

D. Hanahan and Robert A. Weinberg, “Hallmarks of Cancer: The Next Generation,” Cell 144(5), 646-674 (2011).
N. Sethi and Y. Kang, “Unravelling the complexity of metastasis—molecular understanding and targeted therapies,” Nature Reviews Cancer, 1-14 (2011).
J. H. Jang, C. B. Rives, and L. D. Shea, “Plasmid delivery in vivo from porous tissue-engineering scaffolds: transgene expression and cellular transfection,” Mol Ther 12(3), 475-483 (2005).
R. F. Gibly, X. Zhang, M. L. Graham, B. J. Hering, D. B. Kaufman, W. L. Lowe, Jr., and L. D. Shea, “Extrahepatic islet transplantation with microporous polymer scaffolds in syngeneic mouse and allogeneic porcine models,” Biomaterials 32(36), 9677-9684 (2011).
V. Backman and H. K. Roy, “Light-Scattering Technologies for Field Carcinogenesis Detection: A Modality for Endoscopic Prescreening,” Gastroenterology 140(1), 35-+(2011).
O. L. Griffith and J. W. Gray, “′Omic approaches to preventing or managing metastatic breast cancer,” Breast Cancer Res 13(6), 230 (2011).
R. N. Kaplan, B. Psaila, and D. Lyden, “Bone marrow cells in the ‘pre-metastatic niche’: within bone and beyond,” Cancer Metastasis Rev 25(4), 521-529 (2007).
R. N. Kaplan, R. D. Riba, S. Zacharoulis, A. H. Bramley, L. Vincent, C. Costa, D. D. MacDonald, D. K. Jin, K.
Shido, S. A. Kerns, Z. Zhu, D. Hicklin, Y. Wu, J. L. Port, N. Altorki, E. R. Port, D. Ruggero, S. V. Shmelkov, K.
K. Jensen, S. Rafii, and D. Lyden, “VEGFR1-positive haematopoietic bone marrow progenitors initiate the pre-metastatic niche,” Nature 438(7069), 820-827 (2005).
B. Psaila, R. N. Kaplan, E. R. Port, and D. Lyden, “Priming the ‘soil’ for breast cancer metastasis: the pre-metastatic niche,” Breast Dis 26, 65-74 (2006).
M. S. Weiss, B. Penalver Bernabe, A. D. Bellis, L. J. Broadbelt, J. S. Jeruss, and L. D. Shea, “Dynamic, large-scale profiling of transcription factor activity from live cells in 3D culture,” PLoS One 5(11), e14026 (2010).
J. A. Shepard, A. C. Stevans, S. Holland, C. E. Wang, A. Shikanov, and L. D. Shea, “Hydrogel design for supporting neurite outgrowth and promoting gene delivery to maximize neurite extension,” Biotechnol Bioeng (2011).
D. I. Gabrilovich and S. Nagaraj, “Myeloid-derived suppressor cells as regulators of the immune system,” Nat Rev Immunol 9(3), 162-174 (2009).
S. Kusmartsev, S. Nagaraj, and D. I. Gabrilovich, “Tumor-associated CD8+ T cell tolerance induced by bone marrow-derived immature myeloid cells,” J Immunol 175(7), 4583-4592 (2005).
B. Qian, Y. Deng, J. H. Im, R. J. Muschel, Y. Zou, J. Li, R. A. Lang, and J. W. Pollard, “A distinct macrophage population mediates metastatic breast cancer cell extravasation, establishment and growth,” PLoS One 4(8), e6562 (2009).
B. Z. Qian, J. Li, H. Zhang, T. Kitamura, J. Zhang, L. R. Campion, E. A. Kaiser, L. A. Snyder, and J. W. Pollard, “CCL2 recruits inflammatory monocytes to facilitate breast-tumour metastasis,” Nature 475(7355), 222-225 (2011).
G. Francia, W. Cruz-Munoz, S. Man, P. Xu, and R. S. Kerbel, “Mouse models of advanced spontaneous metastasis for experimental therapeutics,” Nat Rev Cancer 11(2), 135-141 (2011).
D. Barkan, J. E. Green, and A. F. Chambers, “Extracellular matrix: a gatekeeper in the transition from dormancy to metastatic growth,” Eur J Cancer 46(7), 1181-1188 (2010).
D. V. Zaykin, L. A. Zhivotovsky, P. H. Westfall, and B. S. Weir, “Truncated product method for combining P-values,” in Genet Epidemiol, (2002), pp. 170-185.
K. A. Janes, J. G. Albeck, S. Gaudet, P. K. Sorger, D. A. Lauffenburger, and M. B. Yaffe, “A systems model of signaling identifies a molecular basis set for cytokine-induced apoptosis,” Science 310(5754), 1646-1653 (2005).
J. H. Jang, D. V. Schaffer, and L. D. Shea, “Engineering biomaterial systems to enhance viral vector gene delivery,” Mol Ther 19(8), 1407-1415 (2011).
J. Yi, J. D. Rogers, I. R. Capoglu, A. Radosevich, Y. Stypula, H. K. Roy, A. Tafove, and V. Backman, “Inverse Scattering Optical Coherence Tomography (ISOCT): quantifying sub-directional tissue mass density correlation function,” Nature Photonics, under review.
H. G. Davies, M. H. F. Wilkins, J. Chayen, and L. F. La Cour, “The use of interference microscope to determine dry mass in living cells and as a quantitative cytochemical method,” Quarterly Journal of Microscopical Science 95(3), 271-304 (1954).
A. J. Radosevich, V. Turzhitsky, N. N. Mutyal, J. D. Rogers, V. Stoyneva, A. K. Tiwari, H. R. Roy, and V. Backman, “Depth-resolved measurement of mucosal microvascular blood content using Low-coherence Enhanced Backscattering Spectroscopy,” Biomedical Optics Express 1(4), 1196-1208 (2010).

1. The method of claim 6, wherein the polymer scaffold does not contain a biological agent prior to the subcutaneous implantation.

2. The method of claim 6, wherein the polymer scaffold is biodegradable.

3. The method of claim 6, wherein the polymer scaffold is bioresorbable.

4. The method of claim 6, wherein the polymer scaffold comprises poly(lactide-co-glycolide).

5. (canceled)

6. A method of detecting metastasis in a subject comprising:
(a) subcutaneously implanting into a subject a biomaterial implant comprising polylactic acid, polyglycolic acid, polycaprolactone, and/or copolymers thereof;
(b) monitoring the biomaterial implant for changes indicative of metastasis.

7. The method of claim 6, wherein the device is implanted into a likely location of metastasis.

8-15. (canceled)

16. The method of claim 6, wherein the biomaterial implant is monitored at multiple timepoints for changes indicative of metastasis.

17. The method of claim 6, wherein the biomaterial implant is monitored daily, semi-daily, weekly, monthly, or yearly for changes indicative of metastasis.

18. The method of claim 6, wherein the biomaterial implant is not removed from the subject to monitor the biomaterial implant for changes indicative of metastasis.

19. The method of claim 6, wherein the biomaterial implant comprises pores.

20. The method of claim 19, wherein the pores are sufficient to permit metastasis of cells into the biomaterial implant.

21. A method comprising:
(a) subcutaneously implanting into a subject a biomaterial implant comprising polylactic acid, polyglycolic acid, polycaprolactone, and/or copolymers thereof, wherein the biomaterial implant comprises pores that are sufficient to permit cells into the biomaterial implant; and
(b) analyzing the biomaterial implant histologically.

22. The method of claim 21, wherein analyzing the biomaterial implant histologically comprises detecting biomarkers on cells collected from the biomaterial implant.

23. A method of collecting cells from a subject comprising:
(a) subcutaneously implanting into a subject a biomaterial implant comprising polylactic acid, polyglycolic acid, polycaprolactone, and/or copolymers thereof, wherein the biomaterial implant comprises pores that are sufficient to permit cells into the biomaterial implant; and
(b) collecting cells from the biomaterial implant.
